(19) United States
(12) Patent Application Publication
(10) Pub. No.: US 2010/0076347 A1
(43) Pub. Date: Mar. 25, 2010

(54) Dynamic movement analysis system

(76) Inventors: Michael J. McGrath, Clonee (IE); Terry Dishongh, Portland, OR (US)

Correspondence Address: TROP, PRUNER & HU, P.C., 1616 S. VOSS RD., SUITE 750, HOUSTON, TX, 77057-2631, US

(21) Appl. No.: 12/284,792

(22) Filed: Sep. 25, 2008

(51) Int. Cl. A61B 5/103

(52) U.S. Cl. 600/595

(57) ABSTRACT

A movement analysis system may use a force sensing mat to record a pattern of applied force from the user's feet as the user undergoes a series of directed movements. The movements may be directed by the system through indicator lights in the mat and/or directions provided on a display screen. The user's response to these directions may be recorded and analyzed to derive indications of the best exercise regime based on the user's capabilities.

### BACKGROUND

This relates generally to electronic systems for monitoring and analyzing human movement.

Persons with injuries, disabilities, and age related infirmities may need targeted exercise programs commensurate with their abilities. In addition, these people may need to be monitored and analyzed to determine the extent of their limitations and to facilitate the design of programs to improve their fitness, range of movement, or coordination.

### BRIEF DESCRIPTION OF THE DRAWINGS

FIG. 1 is a top plan schematic depiction of one embodiment of the present invention;

FIG. 2 is a flow chart for one embodiment of the present invention; and

FIG. 3 is a network depiction according to one embodiment.

### DETAILED DESCRIPTION

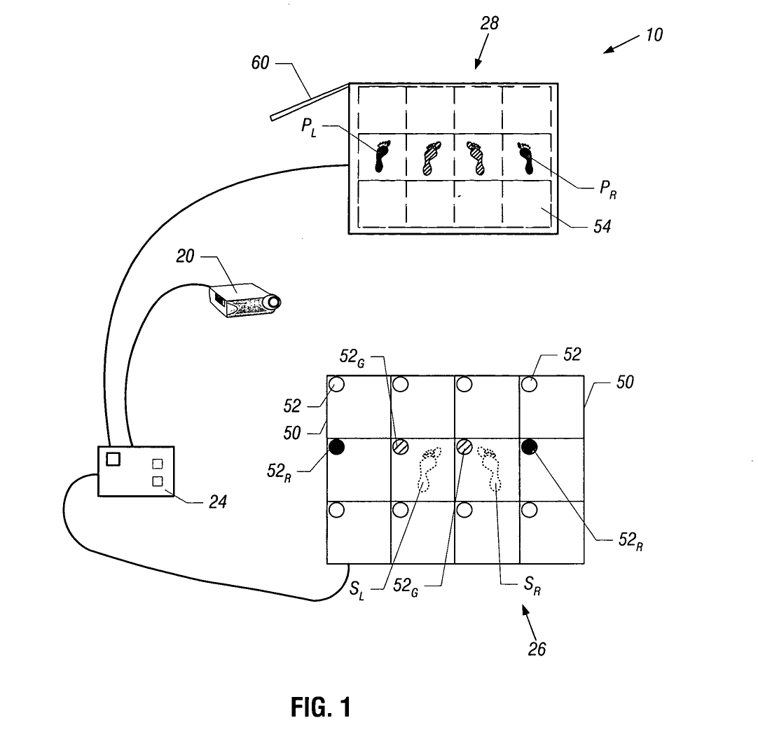

Referring to FIG. 1, an analysis system 10 may include a floor mat 26. The floor mat 26 may be broken up into a plurality of rows and columns made up of individual pressure sensitive squares 50. The individual squares 50 include force sensors that detect dynamically the magnitude of an applied force and the nature of the force applied to the mat. The “nature of the force applied to the mat” is intended to refer to the ability to determine information about a surface area that applies force to the square 50. In some embodiments, this information may indicate whether the user's foot is contacting the square over the entire surface of the user's foot or the user's foot is contacting the square with weight that is focused at a particular part of the foot, either front-to-back or side-to-side.

In one embodiment, each of the squares 50 may be made of Kinotex® tactel force sensor, available from Tactex Controls, Inc., Victoria, B.C., Canada. This force sensor provides the information about both the magnitude of the force and the area through which the force is applied, as well as how that pressure is applied dynamically. A tactel force sensor may include a sensor that measures minute displacements due to forces applied to its surface. It may be constructed of plastic fiber embedded in foam. Thus, it may be flexible or rigid and can operate with soft surfaces or from beneath durable wear layers.

A single sensing element, called a taxel, is composed of send-and-receive fiber. A red light at 650 nanometers shines through the transmit fiber to illuminate the foam. An external force compressing the foam increases the intensity of the backscattered of light. The intensity of the light is monitored by a receive fiber. The receive fiber is coupled to a photodiode that measures the light level returned from the received fiber.

A plurality of the squares 50 may form a matrix of any desired size. In one embodiment, each of the squares 50 may be large enough to easily accommodate a normal person's foot within the periphery of the square 50. Thus, as shown in FIG. 1 in dashed lines, a user may stand on two adjacent squares 50. Of course, it is also possible that the user stands on one or the other of his or her feet.

Each of the squares 50 also includes a light indicator 52. The light indicator may include lighting elements that produce two different light colors. For example, the light color green may indicate the current position of the user's feet and a light color red may indicate the proposed position of the user's feet at the completion of a suggested movement.

Thus, in the example depicted in FIG. 1, the user's feet are currently at the positions S_{L} and S_{R}, as indicated by the dashed footprints. The light indicators 52_{G} indicate the current positions of the user's feet, for example, using green light. The proposed movement may be indicated by the light indicators 52_{R}, which, in this case, suggest moving the feet to an outwardly situated pair of squares 50. The proposed movement may be indicated by red light indicators 52_{R}.

The signals produced by the mat 26 in response to the forces applied by the user's feet are provided to a computer or electronic controller 24. The controller 24 may be a computer in one embodiment. The controller 24 is also coupled to a camera 20 that records the user's movements at all times and enables analysis of the user's movements. Thus, the computer may determine what the user is doing in response to movement suggestions provided on a display 28.

The display 28, also coupled to the controller 24, displays a grid matrix which, in one embodiment, corresponds to the matrix embodied in the mat 26. In one embodiment, the display 28 is supported at an angle to the floor by the stand 60.

The grid image depicted on the display 28 may also include images of a plurality of squares 54 that correspond to the real squares 50 on the mat 26. Thus, the user's current foot position (indicated in the hatched footprints in FIG. 1) by the display 28. The hatched footprints may indicate a foot image that is displayed on the display in a distinct color to indicate the user's current position. The footprints P_{L} and P_{R} may be images of footprints in the new position that the user is being asked to assume. Thus, for example, in response to the display 28, the user may move his feet from the positions, more centered on the mat 26, to the peripheral positions indicated by P_{L} and P_{R} and associated with the squares 50, having the illuminated lights 52_{R}.

In this way, the user can be guided through a series of movements. The user's response to these movements can be monitored in two ways. Feedback is received from the camera 20 to indicate what the user is actually doing. For example, the camera may record the information and may use digital image analysis to make a determination of how stable the user is, how quickly and assuredly the user moves, where the user moves, and how the user is moving.

In addition, the pressure sensitive sensors in each square 50 record the pattern of applied forces of the user's foot positions. The sensors may indicate not only where the user has placed the user's foot, but exactly how weight is distributed over the square 50 by the user's foot and whether or not the user's foot is positioned in the correct position suggested by the display 28. The sensors may also indicate how the weight distribution changes over time.

The system may learn how quickly the user is responding and what the user's range of motion is by indicating progressively more extensive movements and seeing whether the user can actually follow those commands with a reasonable effort. The system may then dynamically configure the subsequent patterns of movements in accordance with the exercise the user needs and the user's ability to do that exercise, based on past results recorded by the system 10.

Thus, the system can provide exercise regimes based on patterns of pressure distribution, such as placing the weight on the heel, toes, outside or inside edges of the feet. Exercises can be targeted to specific muscle groups that may be weakened in particular individuals, such as older individuals. In addition, the exercise pattern may be tailored to physical or mental disabilities or illnesses, including those that result in limited range of motion, reduced response time, and inability to remember sequences of instructions.

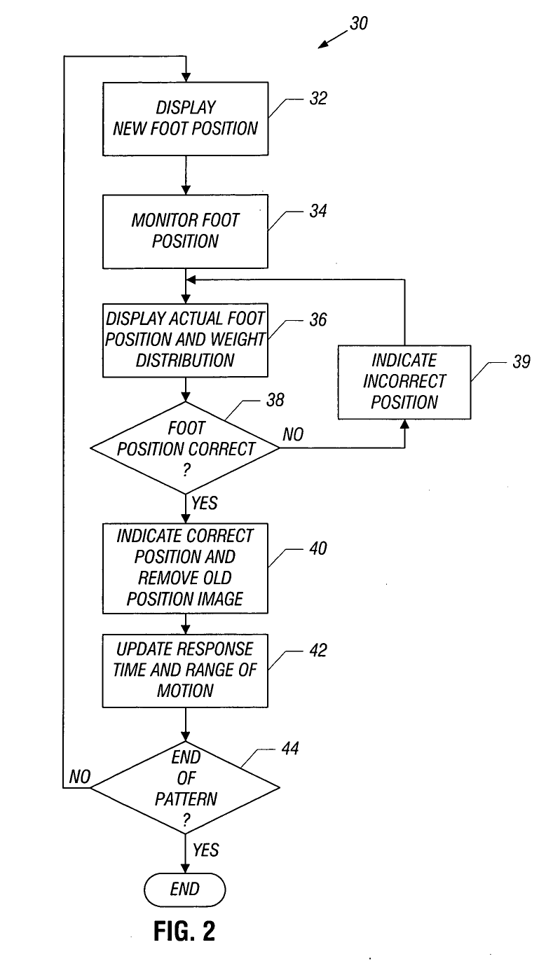

Referring to FIG. 2, in one embodiment, a sequence 30 may be implemented by the controller 24. The sequence 30, in one embodiment, may include software instructions stored in a computer readable medium within the controller 24. Alternatively, those instructions may be embodied in hardware or firmware.

Initially, the sequence 30 may begin by displaying a new foot position, as indicated by the indicators P_{L} and P_{R} on the display 28 in FIG. 1. Then, in block 34, the actual position of the user's feet is monitored. The monitoring may be done through the camera 20 using digital analysis of the resulting image and through the pattern of forces applied to the mat 50, detected through the force sensors within the mat.

Then, the actual foot position of the user is displayed, as indicated in block 36. For example, if the display suggests placement at the positions P_{L} and P_{R}, the user's attempt to comply with that suggested positioning may be indicated by showing where the user's feet actually are, using a different color, such as green, so that the user can then attempt to correct and follow the suggested foot positioning.

In addition, the weight distribution across the user's feet may be indicated by highlighting regions of the foot image displayed on the display screen 28. For example, if the user was asked to place weight on the ball of the left foot, the placement of the user's weight may actually be indicated on the dashed line image to indicate exactly where the weight is placed. The user may have been instructed to place the user's weight primarily on the ball of his foot, in this example, by highlighting the image of the ball of the foot.

Then, a check at diamond 38 determines whether the actual foot positions are correct relative to the instructions that were given or the display 28. If not, the incorrect position is displayed (block 39) and the flow iterates. If the positions are correct, then the flow moves to block 40. In block 40, an indication of the correct position is provided.

In other words, the user is given feedback to indicate that the user has complied with the suggested motion course. The image of the old position of the feet is then removed from the display 28. For example, if the user correctly places the user's feet at the positions P_{L} and P_{R}, those may change color to the color green, in this example, and the two foot images inbound of the images P_{L} and P_{R} may be removed from the display screen.

The user's response time and range of motion is recorded in block 42. This information is then used to design the next stage of the exercise or analysis routine. For example, based on the response time, the amount of time given for the user to respond to the next sequence of instructions may be changed, either increased or decreased, and the extent of movement that is requested may be changed as well by either increasing or decreasing the range of motion, as appropriate.

Algorithms within the controller 24 implement the new sequence. Thereafter, if the end of the pattern of movement is not detected in diamond 44, a new foot position is displayed, as described already. Namely, the desired foot position is indicated on the display 28 using appropriate foot images in one embodiment. In addition, the light indicators on the mat 50 indicate where to put the user's feet for the next sequence or step.

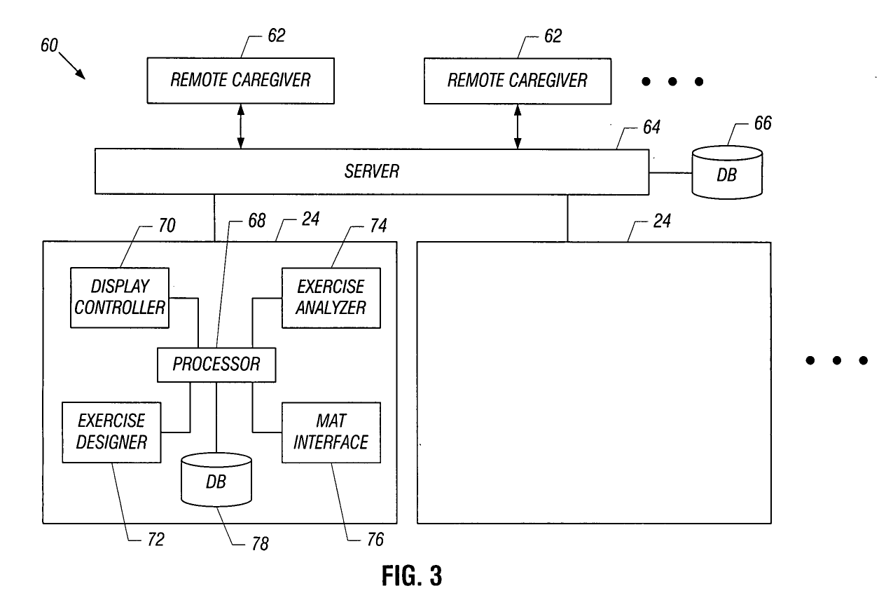

Referring to FIG. 3, in some embodiments, a controller 24 may be linked to a network 60. In some cases, a plurality of controllers 24 associated with different patients, users, or facilities may be linked over a suitable network through a server 64. The network 64 may be a local area network or the Internet, to mention two examples. Thus, a plurality of controllers 24 associated with systems at different locations may be linked through a server 64 to a plurality of care givers 62.

The care givers 62 may be associated with processor-based systems. For example, care givers may be exercise physiologists, doctors, physical therapists, nurses, or hospital technicians, to mention a few examples. Thus, each controller 24 may provide an output over the network to a care giver at a remote location to indicate whether or not the exercise regime is being attempted, how often, and how well the user is doing in attempting to comply with the regime. This information may provide the care giver with immediate feedback, which the care giver may then use to provide directions to modify the routine or to contact the patient or user. Exercise regimen modifications or user contacts may be implemented by messages initiated at the remote care givers 62 and forwarded to a controller 24. Those messages may even be displayed on the display 28.

Thus, in some cases, the care giver can monitor the exercise routine in real time and may provide real time feedback, including encouragement to a remote user. This would enable users to implement the system in their own home under the general guidance of a suitable care giver. In addition, it would enable the care giver to provide inputs to a wide variety of users at a wide variety of disbursed locations.

In some embodiments, the controller 24 may be a processor-based system including a processor 68 coupled to a database 78. The database 78 may include information about suitable exercise regimes, and information correlating particular types of exercises to a particular patient or user problems. The database 78 may be consulted by the exercise designer 72 to come up with an exercise regime based on the user's performance in existing exercises or based on inputs from a care giver, for example. The display controller 70 may be responsible for controlling the display 28. The exercise analyzer 74 may analyze the performance of the user and may provide feedback to the processor for exercise design and modification. That interface 76 interfaces to the mat 26 and provides signals to the mat and receives signals from the mat.

References throughout this specification to “one embodiment” or “an embodiment” mean that a particular feature, structure, or characteristic described in connection with the embodiment is included in at least one implementation encompassed within the present invention. Thus, appearances of the phrase “one embodiment” or “in an embodiment” are not necessarily referring to the same embodiment. Furthermore, the particular features, structures, or characteristics may be instituted in other suitable forms other than the particular embodiment illustrated and all such forms may be encompassed within the claims of the present application.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. a method comprising:
providing a mat to sense the nature of force applied by a user's foot to a floor mat; and
providing an indicator to guide the user through a series of movements based on the nature of the force applied to the mat.

2. The method of claim 1 wherein providing an indicator includes providing a light in said mat to indicate where the user should move the user's foot.

3. The method of claim 1 wherein providing an indicator includes providing a display that also indicates the current position of the user's foot.

4. The method of claim 3 including displaying a suggested foot position.

5. The method of claim 1 including monitoring whether the user moves the user's foot to the suggested foot position, determining how long it takes the user to make the movement, and determining the pattern of force applied by the user's foot to the mat.

6. The method of claim 1 including providing a mat with a plurality of squares, each square including sensors to detect the nature of the force applied to the mat.

7. The method of claim 6 including providing said square to sense how the force is distributed across the user's foot.

8. The method of claim 1 including developing an exercise regime based on the detected ability of the user to follow a pattern of movements.

9. The method of claim 8 including providing a first routine in which the user must progress through a series of movements, measuring the user's performance in making those movements, and, based on the user's performance, providing an exercise routine to improve the user's performance.

10. The method of claim 1 including measuring the user's range of motion.

11. An apparatus comprising:
a floor mat including pressure sensors to sense the nature of a force applied to the mat by a user's foot; and
a controller to guide the user through a series of movements on said mat.

12. The apparatus of claim 11 including a display coupled to said controller, said controller to display an indication of the current position of the user's foot.

13. The apparatus of claim 12, said controller to further display a suggested new foot position.

14. The apparatus of claim 11, said mat to determine the distribution of weight applied by the user's foot to the mat.

15. The apparatus of claim 11 including a controller to determine a pattern of movements on said mat, to determine whether the user has made the movements to the indicated positions, and to determine how quickly the user was able to make the pattern of movements.

16. The apparatus of claim 11 including a controller to suggest a series of movements on said mat, to detect the user's ability to undertake those movements and, based on said determination, to develop an exercise routine for the user.

17. The apparatus of claim 11 including indicators in said mat to indicate where the user should place the user's feet.

18. The apparatus of claim 17 wherein said mat includes a plurality of distinct regions, each region associated with an indicator.

19. The apparatus of claim 18 wherein each of said regions includes two differently colored indicator lights to indicate a suggested foot position with one color and an present foot position with another color.

20. The apparatus of claim 11 including a plurality of distinct regions in said mat, each region including two differently colored indicator lights, said indicator lights to indicate current and future foot positions, said apparatus further including a display for use by the user to indicate the user's current and future foot positions.
